(19) United States
(12) Patent Application Publication
(10) Pub. No.: US 2024/0404626 A1
(43) Pub. Date: Dec. 5, 2024

(54) METHODS AND SYSTEMS FOR AUTOMATED CALLING OF COPY NUMBER ALTERATIONS

(71) Applicant: Foundation Medicine, Inc., Boston, MA (US)

(72) Inventors: Bernard FENDLER, Auburndale, MA (US); Jason D. HUGHES, Providence, RI (US)

(73) Assignee: Foundation Medicine, Inc., Boston, MA (US)

(21) Appl. No.: 18/698,746

(22) PCT Filed: Oct. 7, 2022
(86) PCT No.: PCT/US2022/077764

§ 371 Date: Apr. 4, 2024

Related U.S. Application Data
(60) Provisional application No. 63/253,907, filed on Oct. 8, 2021.

(51) Int. Cl. G16B 20/10; G16B 20/20; G16B 30/10

(52) U.S. Cl. CPC G16B 20/10; G16B 20/20; G16B 30/10

(57) ABSTRACT

Methods and systems for automated calling of copy number alterations (CNAs) are described. The methods and systems utilize sequencing-based coverage ratio data, allele fraction data, segmentation data, and copy number model data for one or more gene loci within one or more subgenomic intervals in a sample from a subject to detect amplifications and deletions of gene loci, and apply a number of thresholds and filters to provide automated calling of CNAs with improved reliability while eliminating the need for process-matched controls and manual curation of the sequencing data.

### CROSS-REFERENCE TO RELATED APPLICATIONS

This application claims the priority benefit of U.S. Provisional patent application Ser. No. 63/253,907, filed Oct. 8, 2021, the contents of which are incorporated herein by reference in their entirety.

### FIELD OF THE INVENTION

The present disclosure relates generally to methods and systems for analyzing genomic profiling data, and more specifically to methods and systems for automated calling of copy number alterations using genomic profiling data.

### BACKGROUND

Structural variants (SVs) are large genomic alterations that typically comprise alterations of at least 50 base pairs (bp) in length (Mahmoud, et al. (2019), “Structural variant calling: the long and the short of it”, Genome Biology 20:246). These large genomic alterations can be classified as deletions, duplications, insertions, inversions, and translocations, and describe different combinations of DNA gains, losses, or rearrangements.

Copy number alterations (CNAs) (also referred to as copy number variations (CNVs)) are a subtype of large structural variants that primarily comprise deletions or duplications, and may encompass alterations of up to half a million nucleotides in length. Somatic copy number variations (CNVs) can play a crucial role in the development of many types of cancer (Samadian, et al. (2018), “Bamgineer: Introduction of simulated allele-specific copy number variants into exome and targeted sequence data sets”, PLOS Comput Biol. 14(3): e1006080). The development of next-generation sequencing (NGS) methods have enabled the development of algorithms to computationally infer CNA profiles from a variety of sequencing data sets, including exome and targeted sequence data.

However, existing methods for detecting and calling CNAs based on sequencing data may require a paired normal sample or process-matched control for sequencing coverage normalization, may require extensive manual curation of the sequencing data, may be susceptible to errors introduced by, e.g., sample contamination, and/or may not handle CNA detection and calling well for small deletions and/or CNA events occurring on chromosome X. Thus, there remains a need for improved methods for automated calling of CNAs.

### BRIEF SUMMARY OF THE INVENTION

Disclosed herein are methods and systems for automated calling of copy number alterations (CNAs) that provide for more accurate detection of copy number alterations and that do not require a coverage-normalization sample or manual curation of the sequence data. In particular, the described methods and systems utilize: (i) a coverage normalization procedure using a “panel of normal” approach that provides proper normalization of chromosome X sequence read data that takes gender into account, (ii) segmentation based on a pruned exact linear time (PELT) method customized to use a particular transformation of the coverage ratio data and extended to account for sample contamination, (iii) an iterative sample contamination detection method based on aberrant SNP profiles (determined using a base-substitution noise model and a copy number model profile to identify a contamination signal), (iv) a novel copy number model determination method based on determination of all locally optimal copy number model configurations and prioritization of models (e.g., the copy number model(s) that are most consistent with the sequence read data and are biologically plausible), and/or (v) automated calling of CNAs based both on the specific copy number model(s) and a scan for additional alterations not explicitly included in the overall copy number model.

Disclosed herein are methods comprising: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules, wherein one or more of the plurality of sequencing reads overlap one or more gene loci within one or more subgenomic intervals in the sample; receiving, at one or more processors, sequence read data for the plurality of sequence reads, and based on the sequence read data: determining, using the one or more processors, a ploidy of the sample, coverage ratio data, allele fraction data, segmentation data, and a copy number model for the one or more gene loci within the one or more subgenomic intervals; identifying, using the one or more processors, a plurality of segments based on the segmentation data; determining, using the one or more processors, copy numbers for the plurality of segments based on at least the coverage ratio data, the allele fraction data, the segmentation data, and the copy number model; detecting, using the one or more processors, the presence of an amplification or deletion for a gene locus of the one or more gene loci based on a copy number of a corresponding segment of the plurality of segments; and calling copy number alterations (CNAs) for the one or more gene loci based on the detected amplifications and deletions for the one or more gene loci.

In some embodiments, the method further comprises merging any duplicate amplifications and deletions detected for a gene locus of the one or more gene loci. In some embodiments, the copy number model predicts a copy number for the one or more gene loci based on the coverage ratio data and allele fraction data. In some embodiments, the coverage ratio data further comprises coverage ratio data for single nucleotide polymorphisms (SNPs) and introns associated with the one or more gene loci. In some embodiments, the copy number model also predicts a sample purity and ploidy for the sample. In some embodiments, the copy number model also outputs the segmentation data. In some embodiments, an amplification is detected when the copy number for the corresponding segment is greater than or equal to the ploidy of the sample. In some embodiments, the detection of deletions comprises identifying homozygous deletions of the one or more gene loci in a corresponding segment. In some embodiments, the detection of deletions comprises identifying heterozygous deletions of the one or more gene loci in a corresponding segment. In some embodiments, the detection of deletions comprises identifying partial deletions of the one or more gene loci in a corresponding segment. In some embodiments, the subject is suspected of having or is determined to have a disease. In some embodiments, the disease is cancer. In some embodiments, the method is used for routine testing. In some embodiments, the method is used for prenatal testing. In some embodiments, the method further comprises collecting the sample from the subject. In some embodiments, the sample comprises a tissue biopsy sample, a liquid biopsy sample, or a normal control. In some embodiments, the sample is a tissue biopsy sample and comprises bone marrow. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample. In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the next generation sequencing (NGS) comprises paired end sequencing. In some embodiments, the sequencer comprises a next generation sequencer. In some embodiments, the method further comprises generating, by the one or more processors, a report indicating the called copy number alterations. In some embodiments, the method further comprises transmitting the report to a healthcare provider. In some embodiments, the report is transmitted via a computer network or a peer-to-peer connection.

Also disclosed herein are methods for automated calling of copy number alterations comprising: receiving, at one or more processors, sequence read data for a plurality of sequence reads that overlap one or more gene loci within one or more subgenomic intervals in a sample from a subject, and based on the sequence read data: determining a ploidy of the sample, coverage ratio data, allele fraction data, segmentation data, and a copy number model for the one or more gene loci within the one or more subgenomic intervals; identifying, using the one or more processors, a plurality of segments based on the segmentation data; determining, using the one or more processors, copy numbers for the plurality of segments based on at least the coverage ratio data, the allele fraction data, the segmentation data, and the copy number model; detecting, using the one or more processors, the presence of an amplification or deletion for a gene locus of the one or more gene loci based on a copy number of a corresponding segment of the plurality of segments; and calling copy number alterations (CNAs) for the one or more gene loci based on the detected amplifications and deletions for the one or more gene loci.

In some embodiments, the method further comprises merging any duplicate amplifications and deletions detected for a gene locus of the one or more gene loci. In some embodiments, the method further comprising generating a report comprising the called copy number alterations for the one or more gene loci. In some embodiments, the method further comprising generating a genomic profile for the subject based on the called copy number alterations for the one or more gene loci.

In some embodiments, the coverage ratio data is determined by aligning a plurality of sequence reads that overlap one or more gene loci within one or more subgenomic intervals in the sample and in a control sample to a reference genome, and determining a number of sequence reads that overlap each of the one or more gene loci within the one or more subgenomic intervals in the sample and in the control sample. In some embodiments, the control sample is a paired normal sample, a process-matched control sample, or a panel of normal control sample.

In some embodiments, the allele fraction data is determined by aligning a plurality of sequence reads that overlap one or more gene loci within one or more subgenomic intervals in the sample to a reference genome, detecting a number of alleles present at a gene locus of the one or more gene loci, and determining an allele fraction for at least one of the alleles present at the gene locus.

In some embodiments, the segmentation data is generated by aligning a plurality of sequence reads that overlap one or more gene loci within one or more subgenomic intervals in the sample to a reference genome, and processing the aligned sequence read data, coverage ratio data, and allele fraction data using a pruned exact linear time (PELT) method to determine a number of segments required to account for the aligned sequence read data, wherein each segment has a same copy number.

In some embodiments, the copy number model predicts a copy number for the one or more gene loci based on the coverage ratio data and allele fraction data. In some embodiments, the coverage ratio data further comprises coverage ratio data for single nucleotide polymorphisms (SNPs) and introns associated with the one or more gene loci. In some embodiments, the copy number model also predicts a sample purity and ploidy for the sample. In some embodiments, the copy number model also outputs the segmentation data. In some embodiments, the ploidy for the sample has a value ranging from 1 to 8.

In some embodiments, an amplification is detected when the copy number for the corresponding segment is greater than or equal to the ploidy of the sample. In some embodiments, an amplification is detected when the copy number for the corresponding segment is greater than or equal to the ploidy of the sample plus a first predetermined value. In some embodiments, the first predetermined value is a value ranging from 2 to 500. In some embodiments, the first predetermined value is a value ranging from 2 to 10.

In some embodiments, an amplification is detected when the copy number for the corresponding segment is greater than or equal to the ploidy of the sample plus a second predetermined value and the gene locus is a member of a first predefined set of gene loci. In some embodiments, the second predetermined value is a value ranging from 0 to 500. In some embodiments, the second predetermined value is a value ranging from 2 to 10. In some embodiments, the first predefined set of gene loci comprises one or more druggable gene target loci, prognostic gene loci, oncogene loci, or any combination thereof. In some embodiments, the first predefined set of gene loci comprises the AR and ERBB2 gene loci.

In some embodiments, the detection of deletions comprises identifying homozygous deletions of the one or more gene loci in a corresponding segment. In some embodiments, homozygous deletions are detected by determining a total copy number for a given gene locus that is equal to the sum of the copy numbers for a first allele and a second allele at the gene locus. In some embodiments, the first allele is a major allele and the second allele is a minor allele. In some embodiments, a homozygous deletion is called if the total copy number for a given gene locus is equal to a third predetermined value. In some embodiments, the third predetermined value is about zero.

In some embodiments, the detection of deletions comprises identifying heterozygous deletions of the one or more gene loci in a corresponding segment. In some embodiments, a heterozygous deletion is called if a copy number for a first allele at a given gene locus is equal to a fourth predetermined value, and a copy number for a second allele at the given gene locus in not equal to the fourth predetermined value. In some embodiments, the fourth predetermined value is about zero. In some embodiments, the first allele is a major allele and the second allele is a minor allele.

In some embodiments, the detection of deletions comprises identifying partial deletions of the one or more gene loci in a corresponding segment. In some embodiments, a partial deletion is called for a given gene locus if log 2 ratios (L2Rs) for neighboring gene loci, single nucleotide polymorphisms (SNPs), and introns are significantly different than the log 2 ratio for the gene locus, and the log 2 ratio for the given gene locus is significantly different from a distribution of L2Rs for non-neighboring gene loci, single nucleotide polymorphisms (SNPs), and introns.

In some embodiments, the called CNAs are used to diagnose or confirm a diagnosis of disease in the subject. In some embodiments, the disease is cancer. In some embodiments, the method further comprises selecting a cancer therapy to administer to the subject based on the called CNAs. In some embodiments, the method further comprises determining an effective amount of a cancer therapy to administer to the subject based on the called CNAs. In some embodiments, the method further comprises administering the cancer therapy to the subject based on the called CNAs. In some embodiments, the cancer therapy comprises chemotherapy, radiation therapy, immunotherapy, a targeted therapy, or surgery. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

Disclosed herein are methods for diagnosing a disease, the methods comprising: diagnosing that a subject has the disease based on detection of copy number alterations (CNAs) for one or more gene loci within one or more subgenomic intervals in a sample from the subject, wherein the detected CNAs are determined according to any of the methods disclosed herein.

Disclosed herein are methods of selecting a cancer therapy, the methods comprising: responsive to detecting copy number alterations (CNAs) for one or more gene loci within one or more subgenomic intervals in a sample from a subject, selecting a cancer therapy for the subject, wherein the detected CNAs are determined according to any of the methods disclosed herein.

Disclosed herein are methods of treating a cancer in a subject, comprising: responsive to detecting copy number alterations (CNAs) for one or more gene loci within one or more subgenomic intervals in a sample from a subject, administering an effective amount of a cancer therapy to the subject, wherein the detected CNAs are determined according to any of the methods disclosed herein.

Disclosed herein are methods for monitoring tumor progression or recurrence in a subject, the methods comprising: detecting copy number alterations (CNAs) for one or more gene loci within one or more subgenomic intervals in a first sample obtained from the subject at a first time point according to any of the methods disclosed herein; detecting copy number alterations (CNAs) for one or more gene loci within one or more subgenomic intervals in a second sample obtained from the subject at a second time point; and comparing the CNAs detected in the first sample to the CNAs detected in the second sample, thereby monitoring the tumor progression or recurrence. In some embodiments, the detection of CNAs in the second sample is determined according to any of the method disclosed herein. In some embodiments, the method further comprises adjusting an anti-cancer therapy in response to the tumor progression. In some embodiments, the method further comprises adjusting a dosage of the anti-cancer therapy or selecting a different anti-cancer therapy in response to the tumor progression. In some embodiments, the method further comprises administering the adjusted anti-cancer therapy to the subject. In some embodiments, the first time point is before the subject has been administered an anti-cancer therapy, and wherein the second time point is after the subject has been administered the anti-cancer therapy. In some embodiments, the subject has a cancer, is at risk of having a cancer, is being routine tested for cancer, or is suspected of having a cancer. In some embodiments, the cancer is a solid tumor. In some embodiments, the cancer is a hematological cancer. In some embodiments, the anti-cancer therapy comprises chemotherapy, radiation therapy, immunotherapy, a targeted therapy, or surgery.

In some embodiments, any of the methods disclosed herein further comprise determining called CNAs for the one or more gene loci within the one or more subgenomic intervals, and applying the called CNAs as a diagnostic value associated with the sample. In some embodiments, any of the methods disclosed herein further comprise generating a genomic profile for the subject based on the called CNAs for the one or more gene loci. In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the subject further comprises results from a nucleic acid sequencing-based test. In some embodiments, the method further comprises selecting an anti-cancer agent, administering an anti-cancer agent, or applying an anti-cancer treatment to the subject based on the generated genomic profile.

In some embodiments, the detection of CNAs for one or more gene loci within one or more subgenomic intervals in a sample using any of the disclosed methods is used in making suggested treatment decisions for the subject. In some embodiments, the detection of CNAs for the one or more gene loci within one or more subgenomic intervals in the sample is used in applying or administering a treatment to the subject.

Disclosed herein are systems comprising: one or more processors; and a memory communicatively coupled to the one or more processors and configured to store instructions that, when executed by the one or more processors, cause the system to: receive sequence read data for a plurality of sequence reads that overlap one or more gene loci within one or more subgenomic intervals in a sample from a subject, and based on the sequence read data: determine a ploidy of the sample, coverage ratio data, allele fraction data, segmentation data, and a copy number model for the one or more gene loci within the one or more subgenomic intervals; identify a plurality of segments based on the segmentation data; determine copy numbers for the plurality of segments based on at least the coverage ratio data, the allele fraction data, the segmentation data, and the copy number model; detect the presence of an amplification or deletion for a gene locus of the one or more gene loci based on a copy number of a corresponding segment of the plurality of segments; and call copy number alterations (CNAs) for the one or more gene loci based on the detected amplifications and deletions for the one or more gene loci.

Also disclosed herein are non-transitory computer-readable storage media storing one or more programs, the one or more programs comprising instructions, which when executed by one or more processors of a system, cause the system to: receive sequence read data for a plurality of sequence reads that overlap one or more gene loci within one or more subgenomic intervals in a sample from a subject, and based on the sequence read data: determined a ploidy of the sample, coverage ratio data, allele fraction data, segmentation data, and a copy number model for the one or more gene loci within the one or more subgenomic intervals; identify a plurality of segments based on the segmentation data; determine copy numbers for the plurality of segments based on at least the coverage ratio data, the allele fraction data, the segmentation data, and the copy number model; detect the presence of an amplification or deletion for a gene locus of the one or more gene loci based on a copy number of a corresponding segment of the plurality of segments; and call copy number alterations (CNAs) for the one or more gene loci based on the detected amplifications and deletions for the one or more gene loci.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for automated CNA calling according to one instance of the disclosed methods.

FIG. 2 provides another non-limiting example of a process flowchart for automated CNA calling according to one instance of the disclosed methods.

FIG. 3 provides a non-limiting example of a process flowchart for scanning deletion calls according to one instance of the disclosed methods.

FIG. 4 provides a non-limiting example of a process flowchart of merging duplicate gene calls according to one instance of the disclosed methods.

FIG. 5 provides a non-limiting example of a process flowchart for setting the properties of a gene object corresponding to a gene locus according to one instance of the disclosed methods.

FIG. 6 depicts an exemplary computing device, in accordance with some instances of the systems described herein.

FIG. 7 depicts an exemplary computer system or computer network, in accordance with some instances of the systems described herein.

### DETAILED DESCRIPTION

Disclosed herein are methods and systems for automated calling of copy number alterations (CNAs) that do not require a coverage-normalization sample or manual curation of the sequence data. The described methods and systems utilize: (i) a coverage normalization procedure using a “panel of normal” approach that provides proper normalization of chromosome X sequence read data that takes gender into account, (ii) segmentation based on, e.g., a pruned exact linear time (PELT) method customized to use a particular transformation of the coverage ratio data and extended to account for sample contamination, (iii) an iterative sample contamination detection method based on aberrant SNP profiles (determined using a base-substitution noise model and a copy number model profile to identify a contamination signal), (iv) a novel copy number model determination method based on determination of all locally optimal copy number model configurations and prioritization of models (e.g., the copy number model(s) that are most consistent with the sequence read data and are biologically plausible), and/or (v) automated calling of CNAs based both on the specific copy number model(s) and a scan for additional alterations not explicitly included in the overall copy number model.

In some instances, for example, methods for automated calling of copy number alterations are described that comprise: receiving, at one or more processors, coverage ratio data, allele fraction data, segmentation data, and copy number model data for one or more gene loci within one or more subgenomic intervals in a sample from a subject; determining, using the one or more processors, an amplification for a gene locus of the one or more gene loci based on a copy number of a corresponding segment identified in the segmentation data and a ploidy of the sample; detecting, using the one or more processors, a deletion of a gene locus of the one or more gene loci based on a copy number of a corresponding segment identified in the segmentation data; merging, using the one or more processors, any duplicate amplification and deletion calls for a gene locus of the one or more gene loci; and calling copy number alterations (CNAs) for the one or more gene loci based on the determined amplifications and detected deletions for the one or more gene loci.

The advantages of the disclosed methods and systems over conventional approaches for calling of CNAs include elimination of the need for a process-matched control, elimination of the need for manual curation, improved coverage normalization relative to the use of a matched control in the conventional approach (reduced noise), improved robustness in that the dependence on the quality of a process-matched control is removed; more precise handling of lower purity tumor samples (due to the reduced noise level and improved copy number modeling), and more reproducible CNA calls (e.g., through the elimination of variation that arises from manual curation).

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “about” a number or value refers to that number or value plus or minus 10% of that number or value. The term ‘about’ when used in the context of a range refers to that range minus 10% of its lowest value and plus 10% of its greatest value.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

As used herein, the term “segmentation” (or “sequence segmentation”) refers to a process for partitioning of the sequence read data into a number of non-overlapping segments that cover all sequence read data points, such that each segment of the plurality of segments is as homogeneous as possible and all sequence reads associated with a given segment have the same copy number. In some instances, segmentation may be performed by processing aligned sequence read data (or other sequencing-related data, e.g., coverage data, allele frequency data, etc., derived from the sequence read data) using any of a variety of methods known to those of skill in the art (see, e.g., Braun and Miller (1998), “Statistical methods for DNA sequence segmentation”, Statistical Science 13(2): 142-162). Examples of segmentation methods include, but are not limited to, circular binary segmentation (CBS) methods, maximum likelihood methods, hidden Markov chain methods, walking Markov methods, Bayesian methods, long-range correlation methods, change point methods, or any combination thereof.

As used herein, the term “ploidy” refers to the average copy number for a plurality of gene loci in a tumor sample as determined by a copy number model. In some instances, the “ploidy” of a tumor sample may differ from the number of complete sets of chromosomes in a cell, and hence the number of possible alleles for autosomal genes (i.e., genes located on numbered, non-sex chromosomes), due to the heterogeneity of the tumor sample (i.e., the variation in tumor sample purity).

The section headings used herein are for organizational purposes only and are not to be construed as limiting the subject matter described.

### Methods for Automated CNA Calling

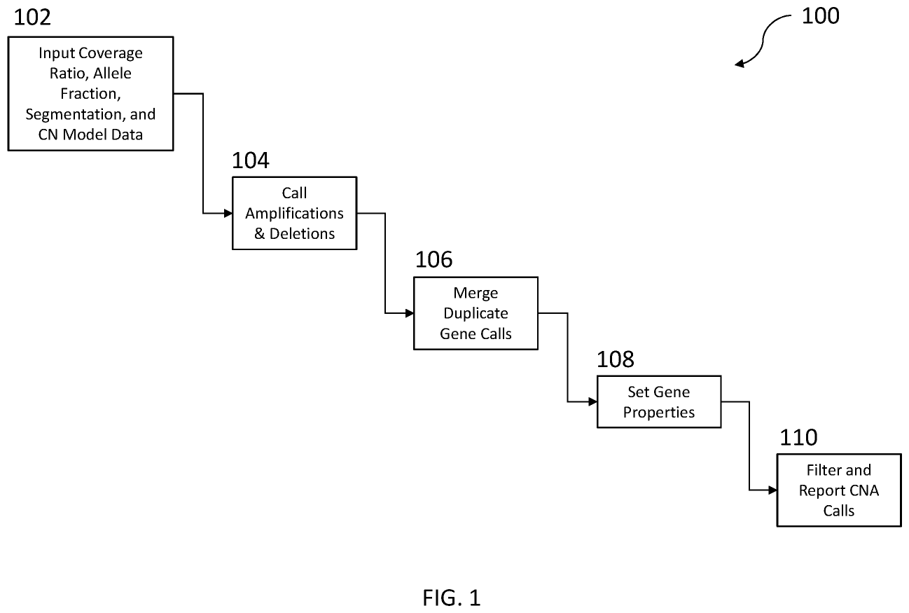

FIG. 1 provides a non-limiting example of a process flowchart for an automated CNA calling process 100 according to one instance of the disclosed methods. The described methods and systems utilize: (i) a coverage normalization procedure using a “panel of normal” approach that provides proper normalization of chromosome X sequence read data that takes gender into account, (ii) segmentation based on, e.g., a pruned exact linear time (PELT) method customized to use a particular transformation of the coverage ratio data and extended to account for sample contamination, (iii) an iterative sample contamination detection method based on aberrant SNP profiles (determined using a base-substitution noise model and a copy number model profile to identify a contamination signal), (iv) a novel copy number model determination method based on determination of all locally optimal copy number model configurations and prioritization of models (e.g., the copy number model(s) that are most consistent with the sequence read data and are biologically plausible), and/or (v) automated calling of CNAs based both on the specific copy number model(s) and a scan for additional alterations not explicitly included in the overall copy number model.

As illustrated in FIG. 1, the automated CNA calling process 100 begins in step 102 with the input of sequencing coverage ratio data (or “coverage ratio data”), allele fraction data, segmentation data, and copy number model data derived by pre-processing of sequence read data for a plurality of sequence reads that overlap one or more gene loci within one or more subgenomic intervals in the sample to be analyzed (e.g., a patient tumor sample).

In some instances, the coverage ratio data for the sample (e.g., a patient tumor sample) is determined by aligning a plurality of sequence reads that overlap one or more gene loci within one or more subgenomic intervals in the sample and in a control (e.g., a paired normal control, a process-matched control, or a “panel of normal” control) to a reference genome (e.g., the GRCh38 human reference genome), and determining a number of sequence reads that overlap each of the one or more gene loci within the one or more subgenomic intervals in the sample and in the control in order to normalize the coverage for the tumor sample to that in the control. In some instances, e.g., if a paired normal control sample is not available, a process-matched control (e.g., a mixture of DNA from a plurality of HapMap cell lines) may be used instead of the paired normal control to normalize coverage. In some instances, e.g., if a paired normal control sample is not available, a “panel of normal” control may be used instead of the paired normal control to normalize coverage.

In some instances, a “panel of normal” (PoN) or “Tangent normalization” control method may be used to normalize sequencing coverage (see, e.g., Tabak, et al. (2019) “The Tangent copy-number inference pipeline for cancer genome analyses”, https://www.biorxiv.org/content/10.1101/566505v1.full.pdf). The Tangent normalization method is a method of normalizing tumor data in order to deal with noise in the data. Specifically, the Tangent method deals with reducing systemic noise resulting from differences in the experimental conditions under which sequencing data from tumors and/or their normal controls were generated. It has been shown that the Tangent normalization method yields a greater reduction in noise than conventional normalization methods.

To begin, let n_{N} be the number of normal non-patient samples (i.e., samples obtained from a plurality of healthy individuals) and n_{T} be the number of tumor samples. Let i be an element of the set {1, 2, . . . , n_{N}} and j be an element of the set {1, 2, . . . , n_{T}}. Define Nᵢ to be the vector of log 2 copy-ratio intensities in genomic order for the i^{th} normal sample. Similarly, define Tⱼ to be the vector of log 2 copy-ratio intensities in genomic order for the j^{th} tumor sample. The normal sample vectors and the tumor sample vectors are elements of the M-dimensional vector space of all possible coverage profiles. Now define a reference subspace N of the vector space of all possible coverage profiles to be the space that contains all linear combinations of the vectors {N₁, N₂, . . . , N_{nN}} of normal samples. N is called the “noise space” and is an (n_{N}−1)-dimensional plane.

Given this setup, the Tangent normalization method proceeds as follows. Start by determining, for each tumor sample vector Tⱼ, the vector in the noise space N that is closest to Tⱼ using a Euclidean metric. Denote this vector p(Tⱼ), the projection of Tⱼ onto N. p(Tⱼ) represents the profile of a normal sample characterized under similar conditions to Tⱼ. The normalization of Tⱼ can now be computed by calculating the difference between Tⱼ and the projection p(Tⱼ) of Tⱼ onto N:

$$Normalization of {T}_{j} = {T}_{j} - p ( {T}_{j} )$$

The projection p(Tⱼ) can be computed directly using standard linear algebra techniques.

The PON method uses the observed patterns of systemic noise in the normal samples to remove typical variation. Chromosome X (chrX) has a specific pattern of half the coverage for gene loci on chrX in males since normal males only have one X chromosome. The PON method thus removes this variation.

In some instances, the allele fraction data for the sample (e.g., a patient tumor sample) is determined by aligning a plurality of sequence reads that overlap one or more gene loci within one or more subgenomic intervals in the sample to a reference genome (e.g., the GRCh38 human reference genome), detecting a number of different alleles present at the one or more gene loci in the one or more subgenomic intervals in the sample, and determining an allele fraction for the different alleles present at the one or more gene loci by dividing the number of sequence reads identified for a given allele sequence by the total number of sequence reads identified for the gene locus.

In some instances, the segmentation data for the sample (e.g., a patient tumor sample) is generated by aligning a plurality of sequence reads that overlap one or more gene loci within one or more subgenomic intervals in the sample to a reference genome (e.g., the GRCh38 human reference genome), and processing the aligned sequence read data (or other sequencing-related data, e.g., coverage ratio data, allele frequency data, etc., derived from the sequence read data) using a segmentation algorithm (e.g., a circular binary segmentation (CBS) method, a maximum likelihood method, a hidden Markov chain method, a walking Markov method, a Bayesian methods, a long-range correlation method, a change point method, or any combination thereof) to generate a plurality of non-overlapping segments such that the sequence associated with a given segment have the same copy number.

In some instances, segmentation may be performed as part of a copy number modeling process to determine a copy number model that best accounts for the coverage ratio and allele fraction data. For example, in some instances, a copy number model may comprise: a purity estimate (e.g., a fraction of cells in the sample that were derived from a tumor), a segmentation (e.g., a division of the genome into components that have undergone either amplifications or losses), and an assignment of copy number state to each segment, where the copy number state is the number of genomic copies of that segment. In some instances, copy number modeling may be facilitated by transforming haploid coverage ratio data (for example, R_{A} and R_{B}, where R_{A} and R_{B} are the haploid coverage ratios for minor and major alleles A and B, respectively) into sum coverage ratio (R_{A}+R_{B}=(2+(C_{A}+C_{B})g)/(1+λg), where C_{A} and C_{B} are the allele counts for the minor and major alleles, A and B, respectively; g=ρ(1−ρ) where ρ is the purity; where λ=(Ψ/2), and where Ψ is the ploidy) and difference coverage ratio (R_{A}−R_{B}=((C_{A}−C_{B})g)/(1+λg)) data for major and minor alleles, and plotting the difference coverage ratio data versus the sum coverage ratio data in a plot that may be overlaid with the segment data and a grid representing allowed copy number states.

In some instances, segmentation may be performed in an iterative manner while simultaneously detecting and correcting for sample contamination in the sequence read data. For example, in some instances, the method may comprise estimating a degree of contamination for the sample based on a distribution of minor allele frequencies for a selected set of heterozygous single nucleotide polymorphisms (SNPs). Then, using the estimated degree of contamination as an initial value for a minor allele frequency (MAF) threshold, the sequencing data is iteratively segmented while simultaneously excluding sequencing data from the segmentation process that comprises SNPs having minor allele frequencies that are below the MAF threshold. At each iteration, the remaining SNPs are classified as aberrant (i.e., likely due to contamination) if they have a minor allele frequency that is different from the MAF for other SNPs detected on the same segment, and the MAF threshold is incrementally adjusted based a comparison of the distribution of aberrant SNP minor allele frequencies to the expected distribution of minor allele frequencies for the selected set of heterozygous SNPs. The segmenting, classifying, and MAF threshold adjusting steps are repeated each time the MAF threshold is increased. When no further increase of the MAF threshold is required (or there is no further change in the distribution of aberrant SNP minor allele frequencies, or a specified maximum number of iterations has been reach), the segmentation data and an estimated degree of contamination for the sample (equal to the final value of the MAF threshold) is output. In some instances, the method further comprises using the segmentation data and estimated degree of contamination to build a copy number model that predicts a copy number for one or more gene loci.

In some instances, the segmentation data for the sample (e.g., a patient tumor sample) may be generated using a pruned exact linear time (PELT) method to determine a number of segments required to properly account for the aligned sequence read data (or other sequencing-related data, e.g., coverage ratio data, allele frequency data, etc., derived from the sequence read data), where each segment (and the sequence reads associated with the segment) has the same copy number. In some instances, the segmentation data is generated using a pruned exact linear time (PELT) method that has been customized to use a particular transformation of the coverage ratio and allele fraction data (e.g., a transformation that enables presentation of the coverage ratio and allele fraction data on the same graph while simultaneously overlaying the predicted copy-number states) and extended to account for sample contamination.

In some instances, a copy number model may be used to identify (or predict) the number of copies of each gene locus, the segmentation of the sample, the sample purity, and the sample ploidy (i.e., an average copy number for the sample) that best account for the measured coverage ratio and allele fraction data for the one or more gene loci (i.e., the one or more gene targets). In some instances, the input data used to generate the copy number model also includes coverage ratio and allele fraction data for single nucleotide polymorphisms (SNPs) and introns. The coverage ratio data is often transformed to log 2 coverage ratio data. Examples of copy number modeling methods include, but are not limited to sliding window methods for computing read count in non-overlapping windows, normalized depth-of-coverage and B allele frequency (i.e., the normalized measure of a relative signal intensity ratio for two alleles) methods, circularized binary segmentation (CBS) methods, statistical analyses of mapping density based on mean-shift approaches, hidden Markov models, read depth-based Bayesian information criteria methods, or any combination thereof (see, e.g., Li and Olivier (2013), “Current analysis platforms and methods for detecting copy number variation”, Physiol. Genomics 45(1):1-16).

In some instances, the input coverage ratio data or copy number estimates used to generate the copy number model are rounded off to integer values. In some instances the output values reported by the finalized copy number model (e.g., predicted copy number values for segments) are integer values. In some instances, the output values reported by the finalized copy number model (e.g., sample purity, sample ploidy, and copy number values predicted for specific gene loci) are real numbers (i.e., continuous). In some instances, sub-clonal events (e.g., sub-clonal deletion events) may occur which do not fit an integer copy number values and may thus have non-integer predicted copy number values.

In some instances, the copy number model may determine that the sample purity (or tumor fraction) has a value ranging from 0.05 to 1.0. In some instances, the determined sample purity may be at least 0.05, at least 0.1, at least 0.2, at least 0.3, at least 0.4, at least 0.5, at least 0.6, at least 0.7, at least 0.8, at least 0.9, at least 0.95, at least 0.98, or at least 0.99. In some instances, the determined sample purity may be at most 0.99, at most 0.98, at most 0.95, at most 0.9, at most 0.8, at most 0.7, at most 0.6, at most 0.5, at most 0.4, at most 0.3, at most 0.2, at most 0.1, or at most 0.05. Any of the lower and upper values described in this paragraph may be combined to form a range included within the present disclosure, for example, in some instances, the determined sample purity may range from 0.1 to 0.8. Those of skill in the art will recognize that the determined sample purity in a given instance may have any value within this range, e.g., about 0.64.

In some instances, the copy number model may determine that the sample ploidy has a value ranging from 1.0 to 10.0. In some instances, the determined sample ploidy may be at least 1.0, at least 2.0, at least 3.0, at least 4.0, at least 5.0, at least 6.0, at least 7.0, at least 8.0, at least 9.0, or at least 10.0. In some instances, the determined sample ploidy may be at most 10.0, at most 9.0, at most 8.0, at most 7.0, at most 6.0, at most 5.0, at most 4.0, at most 3.0, at most 2.0, or at most 1.0. Any of the lower and upper values described in this paragraph may be combined to form a range included within the present disclosure, for example, in some instances, the determined sample ploidy may range from 1.0 to 8.0. Those of skill in the art will recognize that the determined sample ploidy in a given instance may have any value within this range, e.g., about 3.4. In some instances, the sample ploidy may be rounded off and reported as an integer value.

In some instances, the copy number model may predict a copy number for a given gene locus (or segment with which it is associated) ranging from 0 to 500. In some instances, the predicted copy number is at least 0, at least 2, at least 4, at least 6, at least 8, at least 10, at least 20, at least 40, at least 60, at least 80, at least 100, at least 200, at least 300, at least 400, or at least 500. In some instances, the predicted copy number is at most 500, at most 4400, at most 300, at most 200, at most 100, at most 80, at most 60, at most 40, at most 20, at most 10, at most 8, at most 6, at most 4, at most 2, or at most 0. Any of the lower and upper values described in this paragraph may be combined to form a range included within the present disclosure, for example, in some instances, the predicted copy number may range from 1 to 100. Those of skill in the art will recognize that the predicted copy number may have any value within this range, e.g., 7. In some instances, the predicted copy number for a gene locus may be a real valued number rather than an integer.

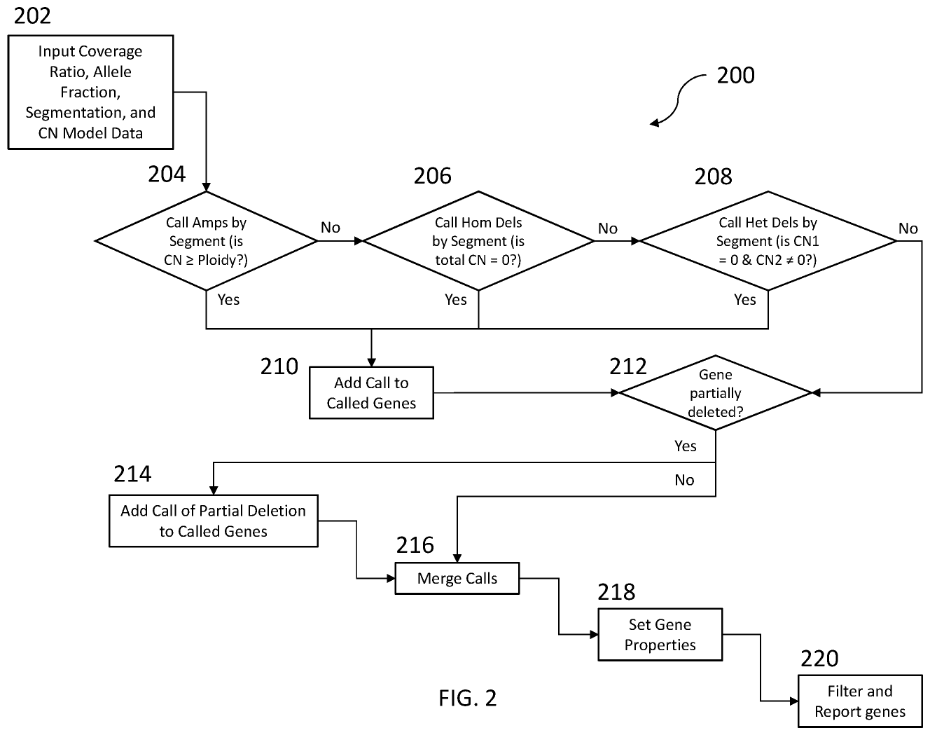

Referring again to FIG. 1, at step 104, amplifications (e.g., increases in the number of copies of a gene locus) or deletions (e.g., deletions of a complete or partial gene locus) of each gene locus of the one or more loci being analyzed are determined on a segment by segment basis. The methods used for detecting amplifications or deletions of the one or more gene loci will be described in more detail below with respect to FIG. 2.

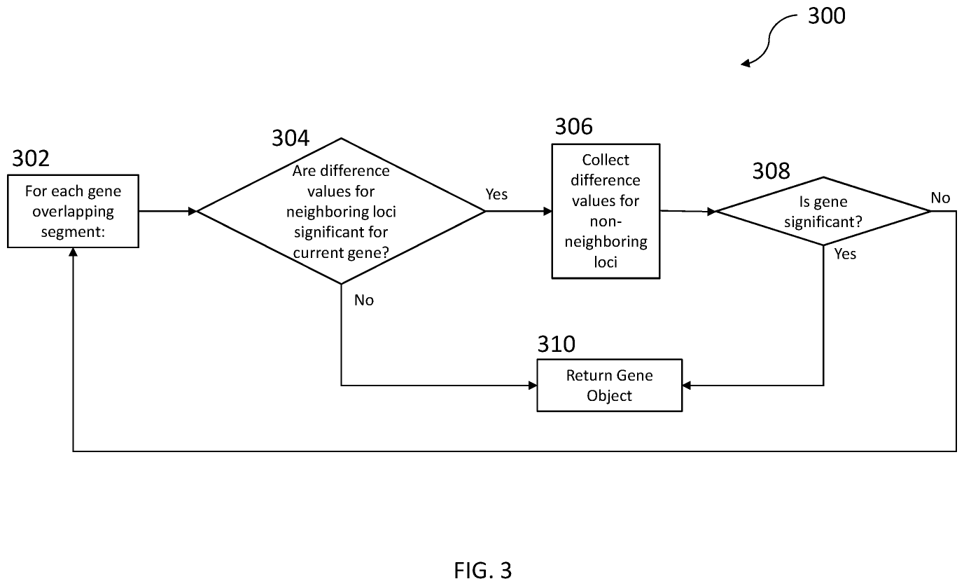

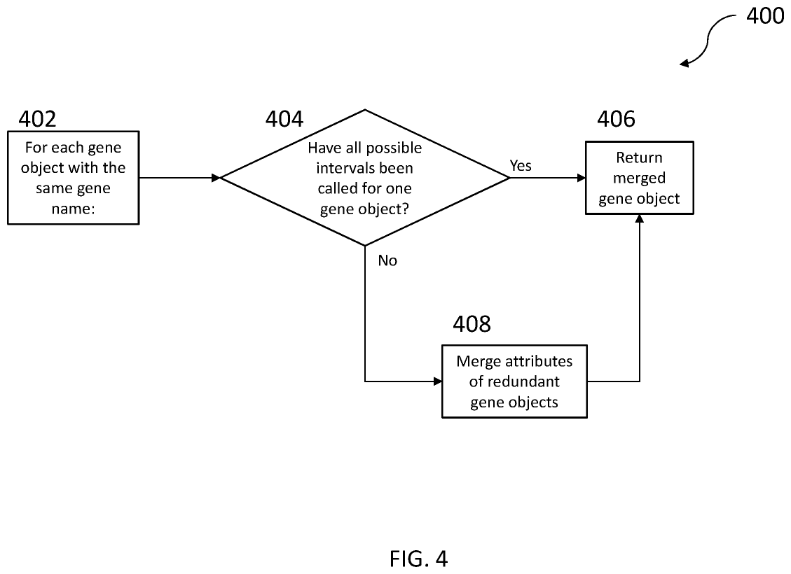

At step 106 of FIG. 1, duplicate gene calls, or more formally, duplicate calls for “gene objects” (i.e., digital data constructs that hold a set of properties (e.g., sequence location, target allele sequences, coverage ratios, etc.) associated with a given gene locus) are merged. Duplicate calls may arise, for example, if a gene sequence is broken into two sub-sequences, and both sub-sequences are called as gene loci comprising amplifications or deletions, thus generating more than one gene object for the locus. In other instances, deletions may be called using both a copy number prediction that comes directly from the copy-number model data and by a partial deletion scanning method (e.g., a method that looks for sequence read(s) that overlap but deviate significantly from the target allele sequence(s) and results in a partial deletion call), in which case more than one gene object is again generated for the locus. Methods for detecting and calling partial deletions will be described in more detail below with respect to FIG. 3. Upon merging, two or more gene objects and their corresponding properties (e.g., sequence location, target allele sequences, coverage ratios, etc.) will be replaced by a single gene object and a consensus set of properties. Methods for merging gene objects and their properties will be described in more detail below with respect to FIG. 4.

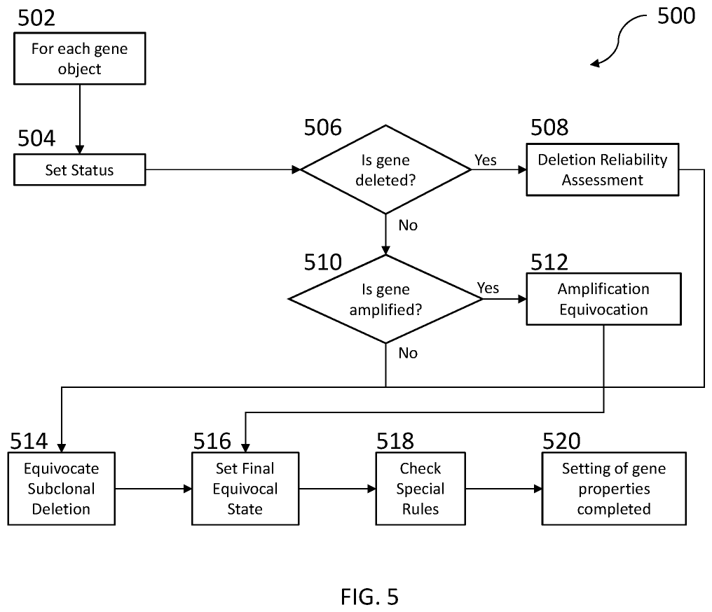

At step 108 in FIG. 1, the set of properties associated with each gene locus (or gene object) is updated. A more detailed description of updating the gene properties will be provided below as part of the description of FIG. 5.

At step 110 of FIG. 1, the CNA results are filtered, e.g., by performing a quality control (QC) procedure for assessing the quality of the sequence read data, the sample purity (e.g., by comparison of a sample purity to a specified sample purity threshold), successful convergence of the copy number model, and/or to assess the reliability of CNA calls for individual gene loci, etc., and prepared for reporting.

FIG. 2 provides a more detailed example of a process flowchart for an automated CNA calling process 200 according to one instance of the disclosed methods. The process begins in step 202 with the input of coverage ratio data, allele fraction data, segmentation data, and copy number model data derived by the pre-processing of sequence read data for a plurality of sequence reads that overlap one or more gene loci within one or more subgenomic intervals in the sample to be analyzed (e.g., a patient tumor sample).

At step 204 in FIG. 2, amplified gene loci are identified on a segment-by-segment basis by comparing the copy number (CN) predicted for a gene locus (or the segment with which it is associated) by the copy number model to the ploidy of the sample as determined by the copy number model. For example, if the copy number of the gene locus (or the segment with which it is associated) is greater than the ploidy in step 204, the gene locus is determined to have been amplified and is added to a list of called genes (or called gene loci) in step 210.

In some instances, the determination of amplification for the gene locus comprises determining if the copy number for the gene locus (or the corresponding segment) is greater than or equal to the ploidy of the sample plus a first predetermined value. In some instances, the first predetermined value may range from 0 to 500. In some instances, the first predetermined value is at least 0, at least 2, at least 4, at least 6, at least 8, at least 10, at least 20, at least 40, at least 60, at least 80, at least 100, at least 200, at least 300, at least 400, or at least 500. In some instances, the first predetermined value is at most 500, at most 4400, at most 300, at most 200, at most 100, at most 80, at most 60, at most 40, at most 20, at most 10, at most 8, at most 6, at most 4, at most 2, or at most 0. Any of the lower and upper values described in this paragraph may be combined to form a range included within the present disclosure, for example, in some instances, the first predetermined value may range from 2 to 10. Those of skill in the art will recognize that the first predetermined value may have any value within this range, e.g., about 11.

In some instances, the determination of amplification for the gene locus in step 204 of FIG. 2 comprises determining if the copy number for the gene locus (or the corresponding segment) is greater than or equal to the ploidy of the sample plus a second predetermined value (i.e., a value that is different than the first predetermined value) and if the gene locus is a member of a first predefined set of gene loci. In some instances, the second predetermined value is a numerical value ranges from 0 to 500. In some instances, the second predetermined value is at least 0, at least 2, at least 4, at least 6, at least 8, at least 10, at least 20, at least 40, at least 60, at least 80, at least 100, at least 200, at least 300, at least 400, or at least 500. In some instances, the second predetermined value is at most 500, at most 4400, at most 300, at most 200, at most 100, at most 80, at most 60, at most 40, at most 20, at most 10, at most 8, at most 6, at most 4, at most 2, or at most 0. Any of the lower and upper values described in this paragraph may be combined to form a range included within the present disclosure, for example, in some instances, the second predetermined value may range from 4 to 60. Those of skill in the art will recognize that the second predetermined value may have any value within this range, e.g., about 7. In some instances, the first predefined set of gene loci comprises one or more druggable gene target loci, prognostic gene loci, oncogene loci, or any combination thereof. In some instances, the first predefined set of gene loci comprises, for example, the AR and ERBB2 gene loci.

At step 206 in FIG. 2, homozygous deletions of gene loci are identified on a segment-by-segment basis by determining a total copy number (total CN) for a given gene locus, and comparing the total copy number of the gene locus to a third predefined value. The total copy number for the gene locus is equal to the sum of the copy numbers for a first allele and a second allele at the gene locus (e.g., a major allele and a minor allele). In some instances, the predicted copy number for a given gene locus may have a negative value due to statistical fluctuations (or noise) in the coverage ratio data input into the copy number model. If the total copy number for the gene locus (or the corresponding segments with which it is associated) is about equal to the third predefined value, a homozygous deletion is called for the gene locus and the gene locus is added to a list of called genes in step 210.

At step 208 in FIG. 2, heterozygous deletions of gene loci are identified on a segment-by-segment basis by comparing the copy numbers for a first allele and a second allele (e.g., a major allele and a minor allele) of a given gene locus to a fourth predefined value. A heterozygous deletion is called for a given gene locus if the copy number of the first allele for the gene locus (or a corresponding segment) is about equal to the fourth predetermined value, and the copy number for the second allele for the gene locus (or a corresponding segment) is not equal to the fourth predetermined value. If the copy number of the first allele for the gene locus (or a corresponding segment) is about equal to the fourth predetermined value, and the copy number for the second allele for the gene locus (or corresponding segment) is not equal to the fourth predetermined value, a heterozygous deletion is called for the gene locus and the gene locus is added to a list of called genes in step 210.

At step 212 in FIG. 2, partial deletions of gene loci may be identified by determining if the log 2 coverage ratios (“log 2 ratios” or “L2Rs”) for neighboring gene loci, single nucleotide polymorphisms (SNPs), and/or introns are significantly different from the L2R for a given gene locus, and if the L2R for the given gene locus is significantly different from a distribution of L2Rs for non-neighboring gene loci, single nucleotide polymorphisms (SNPs), and/or introns. Determination of significant differences in L2Rs will be described in more detail below with respect to FIG. 3. If a partial deletion of a given gene locus is called in step 212, the gene locus is added to a list of called genes in step 214.

At step 216 in FIG. 2, duplicate calls for gene loci (represented digitally as “gene objects”) may be merged. As noted above, duplicate calls may arise, e.g., if a gene sequence is broken into two sub-sequences, and both sub-sequences are called as gene loci comprising amplifications or deletions, or if deletions have been called for the gene locus using both the copy number prediction that comes directly from the copy-number model and by a partial deletion scanning method. Methods for detecting and calling partial deletions will be described in more detail below with respect to FIG. 3. Upon merging, two or more gene objects and their corresponding properties (e.g., sequence location, target allele sequences, coverage ratios, etc.) will be replaced by a single gene object and a consensus set of properties. Methods for merging gene objects and their properties will be described in more detail below with respect to FIG. 4.

At step 218 in FIG. 2, the set of properties associated with each gene locus (or gene object) is updated. A more detailed description of updating the gene properties will be provided below as part of the description of FIG. 5.

At step 220 in FIG. 2, the CNA results are filtered, e.g., by performing a quality control (QC) procedure for assessing the quality of the sequence read data, the sample purity (e.g., by comparison of a sample purity to a specified sample purity threshold), successful convergence of the copy number model, and/or to assess the reliability of CNA calls for individual gene loci, etc., and prepared for reporting.

FIG. 3 provides a non-limiting example of a process (or subroutine) 300 for the system to call partial deletions in gene loci. Starting at step 302, each gene overlapping segment is reviewed for partial deletions. As noted above, partial deletions of gene loci may be identified by determining, at step 304, if the log 2 coverage ratios (“log 2 ratios” or “L2Rs”) for neighboring loci (e.g., gene loci, single nucleotide polymorphisms (SNPs), and/or introns) are significantly different from the L2R for a given gene locus (and correspond to a non-zero copy number), and determining, at step 308, if the L2R for the given gene locus is significantly different from a distribution of L2Rs for non-neighboring loci (e.g., gene loci, single nucleotide polymorphisms (SNPs), and/or introns), where L2Rs for non-neighboring loci are collected at step 306. If the L2Rs for neighboring loci are determined to be not significantly different from the L2R for a gene locus at step 304, that finding is added to the gene object corresponding to the gene locus and returned at step 310 (the partial deletion evaluation process returns to process 200 illustrated in FIG. 2 after all gene overlapping segments have been scanned). If the L2Rs for neighboring loci are determined to be significantly different from the L2R for a gene locus at step 304, and the L2R for the given gene locus is determined to be significantly different from a distribution of L2Rs for non-neighboring loci at step 308, that finding is added to the gene object corresponding to the gene locus and returned at step 310.

In some instances, e.g., if the sample purity is below a specified purity threshold, the log 2 coverage ratios (L2Rs) for neighboring gene loci, single nucleotide polymorphisms (SNPs), and/or introns (i.e., the intervening gene locus, SNP, or intron residing between two other gene loci of interest or adjacent to a gene locus of interest) may be determined to be significantly different from the L2R for a given gene locus at step 304 if: (i) the average copy number for the two nearest neighbor loci (e.g., gene loci, SNP loci, and/or introns) is less than or equal to a first specified threshold and a fractional difference in coverage ratios (e.g., a log 2 coverage ratio). For example, a fractional difference could be defined as |(L2Rᵢ−L2Rᵢ₊₁)|/(max [L2Rᵢ, L2Rᵢ₊₁]), where i is the genomic index of the neighboring loci) for the two nearest neighbor loci is less than or equal to a second specified threshold, or (ii) the predicted copy number at the current gene locus (locus i) is less than or equal to zero (as noted above, predicted copy number values may be negative due to statistical fluctuations in the coverage ratio (or log 2 coverage ratio) data), the predicted copy number at the nearest neighbor locus i+1 (e.g., nearest neighbor gene locus, SNP locus, and/or intron) is less than or equal to zero, and the fractional difference (as defined above) is less than a first specified threshold, and (iii) the two-tailed p-value is less than a third specified threshold. In some instances, the first specified threshold, the second specified threshold, and the third specified threshold may each independently range from about 0.000001 to about 0.3. In some instances, the first specified threshold, the second specified threshold, and the third specified threshold may each independently be at least or about 0.000001 at least or about 0.00001, at least or about 0.0001, at least or about 0.001, at least or about 0.002, at least or about 0.003, at least or about 0.004, at least or about 0.005, at least or about 0.01, at least or about 0.02, at least or about 0.03, at least or about 0.04, at least or about 0.05, at least or about 0.06, at least or about 0.07, at least or about 0.08, at least or about 0.09, at least or about 0.1, at least or about 0.12, at least or about 0.14, at least or about 0.16, at least or about 0.18, at least or about 0.20, at least or about 0.22, at least or about 0.24, at least or about 0.26, at least or about 0.28, or at least or about 0.30.

In some instances, e.g., if the sample purity is above the specified purity threshold, the log 2 coverage ratio of a given gene locus may be determined to be significantly different from a distribution of L2Rs for non-neighboring gene loci, single nucleotide polymorphisms (SNPs), and/or introns at step 304 if the average copy number for the two nearest neighbor loci (e.g., gene loci, SNP loci, and/or introns) is less than a first specified threshold, the fractional difference in log 2 coverage ratios (as defined above) is less than a second specified threshold, and the two-tailed p-value is less than a third specified threshold. In these instances, the first specified threshold, the second specified threshold, and the third specified threshold may each independently range from about 0.000001 to about 0.3. In some instances, the first specified threshold, the second specified threshold, and the third specified threshold may each independently be at least or about 0.000001, at least or about 0.00001, at least or about 0.0001, at least or about 0.001, at least or about 0.002, at least or about 0.003, at least or about 0.004, at least or about 0.005, at least or about 0.01, at least or about 0.02, at least or about 0.03, at least or about 0.04, at least or about 0.05, at least or about 0.06, at least or about 0.07, at least or about 0.08, at least or about 0.09, at least or about 0.1, at least or about 0.12, at least or about 0.14, at least or about 0.16, at least or about 0.18, at least or about 0.20, at least or about 0.22, at least or about 0.24, at least or about 0.26, at least or about 0.28, or at least or about 0.30.

In step 308 of FIG. 3, the L2R for the given gene locus may be determined to be significantly different from a distribution of L2Rs for non-neighboring gene loci, single nucleotide polymorphisms (SNPs), and/or introns if, for example, the two-tailed p-value is less than a specified threshold.

FIG. 4 provides a non-limiting example of a process (or subroutine) 400 for pruning and merging duplicate gene calls. As noted above, duplicate calls may arise, e.g., if a gene sequence is broken into two sub-sequences, and both sub-sequences are called as gene loci comprising amplifications or deletions, or if deletions have been called for the gene locus using both the copy number prediction that comes directly from the copy-number model and by a partial deletion scanning method. The process begins at step 402 by reviewing each gene object with the same gene name. At step 404, a determination is made as to whether or not all corresponding genomic intervals (e.g., sequence reads aligned to a given gene locus) have been called for the same gene object. If so, the gene objected is returned at step 406 (the pruning and merging process returns to process 200 illustrated in FIG. 2 after all gene objects have been reviewed). If not, the duplicate calls are reviewed to determine which call(s) should be pruned (discarded) and which call(s) should be saved and merged. The attributes (e.g., gene target sequence, L2R data, amplification or deletion status, etc.) of the saved duplicate gene objects are merged at step 408, and subsequently returned to the main process at step 406.

Duplicate gene calls to be merged may comprise gene sequences that are identical, overlap, or are subsequences of the complete gene sequence. For example, the pruning step is frequently performed when one gene object spans the entire gene sequence and another gene object for the same gene is a subsequence.

FIG. 5 provides a non-limiting example of a process 500 for setting or updating the properties of a gene object corresponding to a gene locus. The process begins in step 502 with a review of each gene object. In step 504, a status is set for the gene object (e.g., type of gene, relevance of an alteration given the size of a copy number event, reliability of an amplification or deletion call, whether the gene is known or unknown, etc.).

In step 506 of FIG. 5, the gene object is assessed to determine if a deletion call has been made for the gene. If so, a reliability assessment for the deletion call is made at step 508. If not, the gene object is assessed at step 510 to determine if an amplification call has been made for the gene. If so, a reliability assessment (amplification equivocation assessment) for the amplification call is made at step 512. If not, a reliability assessment for a subclonal deletion is made at step 514.

The deletion call reliability assessment made in step 508 of FIG. 5 may comprise determining if a bulk purity for the sample (i.e., a parameter determined by the copy number model that characterizes the bulk tumor mass of the sample, or in other words, a copy number value that covers the range of copy numbers exhibited by the tumor tissue in the sample), and assigning a quality control status of true or false for each gene object for which a deletion was called based on a comparison of the bulk purity to a first specified bulk purity threshold.

The amplification equivocation assessment made in step 512 of FIG. 5 may comprise setting a status for a given gene locus of the one or more gene loci as amplification equivocal if the copy number of a corresponding segment to which the gene locus maps is less than or equal to a ploidy of the sample plus a first specified ploidy difference threshold.

In some instances, the amplification equivocation assessment made in step 512 of FIG. 5 may comprise setting a status for a given gene locus of the one or more gene loci as amplification equivocal if the copy number of a corresponding segment to which the gene locus maps is less than or equal to a ploidy of the sample plus a second specified ploidy difference threshold and the gene locus is not included in a third predefined set of gene loci that are called at copy numbers below a second specified copy number threshold.

In some instances, the amplification equivocation assessment made in step 512 of FIG. 5 may comprise setting a status for a given gene locus of the one or more gene loci as amplification equivocal if the copy number of a corresponding segment to which the gene locus maps is equal to a ploidy of the sample plus a third specified ploidy difference threshold and the gene locus is included in a fourth predefined set of gene loci.

In some instances, the amplification equivocation assessment made in step 512 of FIG. 5 may comprise setting a status for a given gene locus of the one or more gene loci as amplification equivocal if the copy number of a corresponding segment to which the gene locus maps is equal to a ploidy of the sample plus a fourth specified ploidy difference threshold and the gene locus is included in a fifth predefined set of gene loci.

In some instances, the first specified ploidy difference threshold, the second specified ploidy difference threshold, the third specified ploidy difference threshold, and the fourth specified ploidy difference threshold may each independently range from 1 to 12 (e.g., an integer or floating point number). In some instances, the first specified ploidy difference threshold, the second specified ploidy difference threshold, the third specified ploidy difference threshold, and the fourth specified ploidy difference threshold are each independently at least 1, at least 2, at least 3, at least 4, at least 5, at least 6, at least 7, at least 8, at least 9, at least 10, at least 11, or at least 12. In some instances, the first specified ploidy difference threshold, the second specified ploidy difference threshold, the third specified ploidy difference threshold, and the fourth specified ploidy difference threshold are each independently at most 12, at most 11, at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some instances, the first specified ploidy difference threshold, the second specified ploidy difference threshold, the third specified ploidy difference threshold, and the fourth specified ploidy difference threshold may each independently range from 2 to 11.

In some instances, the second specified copy number threshold ranges from 2 to 12. In some instance, the second specified copy number threshold is at least 2, at least 3, at least 4, at least 5, at least 6, at least 7, at least 8, at least 9, at least 10, at least 11, or at least 12. In some instances, the second specified copy number threshold is at most 12, at most 11, at most 10, at most 9, at most 8, at most 7, at most 6, at most 5, at most 4, at most 3, or at most 2. Any of the lower and upper values described in this paragraph may be combined to form a range included within the present disclosure, for example, in some instances, the second specified copy number threshold may range from 3 to 7.

In some instances, the third predefined set of gene loci, the fourth predefined set of gene loci, and the fifth predefined set of gene loci may each independently comprise the ERBB2 gene locus, the AR gene locus, or any combination thereof.

In step 518 of FIG. 5, a reliability assessment for a subclonal deletion call is made. In some instances, a status of “subclonal deletion equivocal” is set if a deletion has been called for the gene locus, the copy number model converged successfully, the sample purity is greater than a specified purity threshold, a product of a first separation coefficient and a separation is less than a coverage ratio difference, and the coverage ratio difference is less than a product of a second separation coefficient and the separation. The separation may be a copy number model parameter based on sample purity and ploidy; e.g., separation β₁=ρ(Ψρ+2(1−ρ)), where ρ is the sample purity (tumor fraction) and Ψ is the sample ploidy. The coverage ratio difference may be equal to the sum of the coverage ratios for major and minor alleles (Rₐ+R_{b}; equal to twice the mean coverage ratio) minus a zero level (or ground state) parameter for the copy number model, β₀=(2(1−ρ))/(Ψρ+2(1−ρ)).

In some instances, the specified purity threshold ranges from 0 to 1. In some instances, the specified purity threshold is at least 0, at least 0.05, at least 0.1, at least 0.2, at least 0.3, at least 0.4, at least 0.5, at least 0.6, at least 0.7, at least 0.8, at least 0.9, or at least 0.95. In some instances, the specified purity threshold is at most 1, at most 0.95, at most 0.9, at most 0.8, at most 0.7, at most 0.6, at most 0.5, at most 0.4, at most 0.3, at most 0.2, at most 0.1, at most 0.05. Any of the lower and upper values described in this paragraph may be combined to form a range included within the present disclosure, for example, in some instances, the second specified purity threshold may range from 0.1 to 0.7.

In some instances, the first separation coefficient ranges from 0.10 to 0.30. In some instances, the first separation coefficient is at least 0.1, at least 0.15, at least 0.2, at least 0.25, or at least 0.3. In some instances, the first separation coefficient is at most 0.3, at most 0.25, at most 0.2, at most 0.15, or at most 0.1. Any of the lower and upper values described in this paragraph may be combined to form a range included within the present disclosure, for example, in some instances, the first separation coefficient may range from 0.15 to 0.25.

In some instances, the second separation coefficient ranges from 0.50 to 0.90. In some instances, the second separation coefficient is at least 0.5, at least 0.6, at least 0.7, at least 0.8, or at least 0.9. In some instances, the second separation coefficient is at most 0.9, at most 0.8, at most 0.7, at most 0.6, or at most 0.5. Any of the lower and upper values described in this paragraph may be combined to form a range included within the present disclosure, for example, in some instances, the second separation coefficient may range from 0.6 to 0.8.

At step 516 in FIG. 5, a final status of “equivocal” may be set for a gene locus if an amplification or deletion call was determined to be equivocal as described above. At step 518, a set of special rules (e.g., a list of specific gene loci for which the disclosed CNA calling procedures are known to underperform) may be consulted so that calls for the listed gene loci may be filtered out. At step 520, the process of updating status settings for the one or more gene loci is complete.

In some instances, the disclosed methods for automated calling of CNAs may be applied to sequence read data covering a panel of gene loci comprising at least 10, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, at least 300, at least 320, at least 340, at least 360, at least 380, at least 400, or more than 400 gene loci. In some instances, the panel may further comprise a plurality of genome-wide SNP loci, e.g., comprising at least 100, at least 200, at least 300, at least 400, at least 500, at least 600, at least 700, at least 800, at least 900, at least 1,000, at least 2,000, at least 3,000, at least 4,000, at least 5,000, at least 600, at least 7,000, at least 8,000, at least 9,000, or at least 10,000 SNP loci. In some instances, the panel may comprise at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 5,500, at least 6,000, at least 6,500, at least 7,000, at least 7,500, at least 8,000, at least 8,500, at least 9,000, at least 9,500, at least 10,000, at least 11,000, at least 12,000, at least 13,000, at least 14,000, or at least 15,000 target loci comprising a combination of gene loci, SNP loci, exon loci, intron loci, or any combination thereof.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for automated detection and calling of copy number alterations (CNAs) may be used to diagnose the presence of disease or other conditions (e.g., cancer, genetic disorders (such as Down Syndrome and Fragile X), neurological disorders, or any other disease type where copy number is relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for automated CNA calling may be used to predict genetic disorders in fetal DNA. (e.g., for invasive or non-invasive prenatal testing). For example, sequence read data obtained sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify copy number alterations associated with, e.g., Down Syndrome (trisomy 21), trisomy 18, trisomy 13, and extra or missing copies of the X and Y chromosomes.

In some instances, the disclosed methods for automated CNA calling may be used to select a subject (e.g., a patient) for a clinical trial based on the CNA value determined for one or more gene loci. In some instances, patient selection for clinical trials based on, e.g., identification of CNAs at one or more gene loci, may accelerate the development of targeted therapies and improve the healthcare outcomes for treatment decisions.

In some instances, the disclosed methods for automated detection and calling of copy number alterations (CNAs) may be used to select an appropriate therapy or treatment (e.g., a cancer therapy or cancer treatment) for a subject. In some instances, for example, the cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the disclosed methods for automated detection and calling of copy number alterations (CNAs) may be used in treating a disease (e.g., a cancer) in a subject. For example, in response to determining that CNAs occur in one or more gene loci in a patient sample using any of the methods disclosed herein, an effective amount of a cancer therapy or cancer treatment may be administered to the subject.

In some instances, the disclosed methods for automated detection and calling of copy number alterations (CNAs) may be used for monitoring disease progression or recurrence (e.g., cancer or tumor progression or recurrence) in a subject. For example, in some instances, the methods may be used to detect CNAs in a first sample obtained from the subject at a first time point, and used to detect CNAs in a second sample obtained from the subject at a second time point, where comparison of the first determination of CNAs and the second determination of CNAs allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., a cancer treatment or cancer therapy) for a subject, e.g., by adjusting a treatment dose and/or selecting a different treatment in response to a change in called copy number alterations (CNAs).

In some instances, called CNAs determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for automated detection and calling of copy number alterations (CNAs) may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for automated detection and calling of copy number alterations (CNAs) as part of a genomic profiling process (or inclusion of the output from the disclosed methods for called CNAs as part of the genomic profile of the subject) can improve the validity of, e.g., disease detection calls and treatment decisions, made on the basis of the genomic profile by, for example, independently confirming the presence of CNAs in one or more gene loci in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples include, but are not limited to, a tumor sample, a tissue sample, a biopsy sample, a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly (A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly (A) tail of mature mRNAs by priming with oligo (dT)-containing oligonucleotides. Methods for depletion, poly (A) enrichment, and cDNA synthesis are well known to those of skill in the art.

In some instances, the sample may comprise a tumor content, e.g., comprising tumor cells or tumor cell nuclei. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g. a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with a cancer therapy (or cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with a cancer therapy (or cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164 (1): 35-42; Masuda, et al., (1999) Nucleic Acids Res. 27 (22): 4436-4443; Specht, et al., (2001) Am J Pathol. 158 (2): 419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4 (11): 903-5; Hodges, E. et al. (2007) Nat. Genet. 39 (12): 1522-7; and Okou, D. T. et al. (2007) Nat. Methods 4 (11): 907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing”, and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (1970) “A General Method Applicable to the Search for Similarities in the Amino Acid Sequence of Two Proteins”, J. Molecular Biology 48 (3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26 (5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85 (6): 847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation-detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1 e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9): 1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix—Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems for Automated CNA Calling

Also disclosed herein are systems designed to implement any of the disclosed methods for automated detection and calling of CNAs in one or more gene loci in a sample from a subject. The systems may comprise, e.g., one or more processors, and a memory unit communicatively coupled to the one or more processors and configured to store instructions that, when executed by the one or more processors, cause the system to: receive, at one or more processors, coverage ratio data, allele fraction data, segmentation data, and copy number model data for one or more gene loci within one or more subgenomic intervals in a sample from a subject; determine, using the one or more processors, an amplification for a gene locus of the one or more gene loci based on a copy number of a corresponding segment identified in the segmentation data and a ploidy of the sample; detect, using the one or more processors, a deletion of a gene locus of the one or more gene loci based on a copy number of a corresponding segment identified in the segmentation data; merge, using the one or more processors, any duplicate amplification and deletion calls for a gene locus of the one or more gene loci; and call copy number alterations (CNAs) for the one or more gene loci based on the determined amplifications and detected deletions for the one or more gene loci.

In some instances, the disclosed systems may further comprise a sequencer, e.g., a next generation sequencer (also referred to as a massively parallel sequencer). Examples of next generation (or massively parallel) sequencing platforms include, but are not limited to, the Roche 454, Illumina Solexa, ABI-SOLID, ION Torrent, or Pacific Bioscience sequencing platforms.

In some instances, the disclosed systems may be used for automated detection and calling of CNAs in any of a variety of samples as described herein (e.g., a tissue sample, biopsy sample, hematological sample, or liquid biopsy sample derived from the subject).

In some instances, the plurality of gene loci for which sequencing data is processed to determine copy number alterations may comprise at least 1, 2, 3, 4, 5, 6, 7, 8, 9, 10, 20, 30, 40, 50, 60, 70, 80, 90, 100, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of copy number alterations in one or more gene loci is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

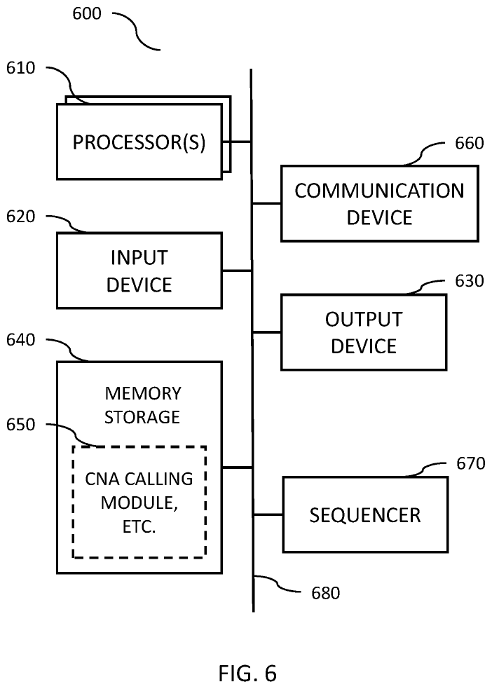

FIG. 6 illustrates an example of a computing device or system in accordance with one embodiment. Device 600 can be a host computer connected to a network. Device 600 can be a client computer or a server. As shown in FIG. 6, device 600 can be any suitable type of microprocessor-based device, such as a personal computer, workstation, server or handheld computing device (portable electronic device) such as a phone or tablet. The device can include, for example, one or more processor(s) 610, input devices 620, output devices 630, memory or storage devices 640, communication devices 660, and nucleic acid sequencers 670. Software 650 residing in memory or storage device 640 may comprise, e.g., an operating system as well as software for executing the methods described herein. Input device 620 and output device 630 can generally correspond to those described herein, and can either be connectable or integrated with the computer.

Input device 620 can be any suitable device that provides input, such as a touch screen, keyboard or keypad, mouse, or voice-recognition device. Output device 630 can be any suitable device that provides output, such as a touch screen, haptics device, or speaker.

Storage 640 can be any suitable device that provides storage (e.g., an electrical, magnetic or optical memory including a RAM (volatile and non-volatile), cache, hard drive, or removable storage disk). Communication device 660 can include any suitable device capable of transmitting and receiving signals over a network, such as a network interface chip or device. The components of the computer can be connected in any suitable manner, such as via a wired media (e.g., a physical system bus 680, Ethernet connection, or any other wire transfer technology) or wirelessly (e.g., Bluetooth®, Wi-Fi®, or any other wireless technology).

Software module 650, which can be stored as executable instructions in storage 640 and executed by processor(s) 610, can include, for example, an operating system and/or the processes that embody the functionality of the methods of the present disclosure (e.g., as embodied in the devices as described herein).

Software module 650 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 640, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

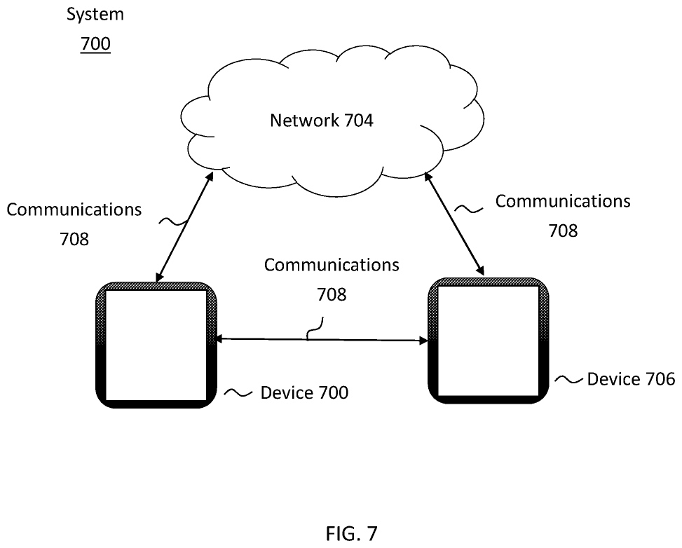

Device 600 may be connected to a network (e.g., network 704, as shown in FIG. 7 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 600 can be implemented using any operating system, e.g., an operating system suitable for operating on the network. Software module 650 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 610.

Device 600 can further include a sequencer 670, which can be any suitable nucleic acid sequencing instrument.

FIG. 7 illustrates an example of a computing system in accordance with one embodiment. In system 700, device 600 (e.g., as described above and illustrated in FIG. 6) is connected to network 704, which is also connected to device 706. In some embodiments, device 706 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio RS system.

Devices 600 and 706 may communicate, e.g., using suitable communication interfaces via network 704, such as a Local Area Network (LAN), Virtual Private Network (VPN), or the Internet. In some embodiments, network 704 can be, for example, the Internet, an intranet, a virtual private network, a cloud network, a wired network, or a wireless network. Devices 600 and 706 may communicate, in part or in whole, via wireless or hardwired communications, such as Ethernet, IEEE 802.11b wireless, or the like. Additionally, devices 600 and 706 may communicate, e.g., using suitable communication interfaces, via a second network, such as a mobile/cellular network. Communication between devices 600 and 706 may further include or communicate with various servers such as a mail server, mobile server, media server, telephone server, and the like. In some embodiments, Devices 600 and 706 can communicate directly (instead of, or in addition to, communicating via network 704), e.g., via wireless or hardwired communications, such as Ethernet, IEEE 802.11b wireless, or the like. In some embodiments, devices 600 and 706 communicate via communications 708, which can be a direct connection or can occur via a network (e.g., network 704).

One or all of devices 600 and 706 generally include logic (e.g., http web server logic) or are programmed to format data, accessed from local or remote databases or other sources of data and content, for providing and/or receiving information via network 704 according to various examples described herein.

### Exemplary Implementations

Exemplary implementations of the methods and systems described herein include:

1. A method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules, wherein one or more of the plurality of sequencing reads overlap one or more gene loci within one or more subgenomic intervals in the sample;
receiving, at one or more processors, sequence read data for the plurality of sequence reads, and based on the sequence read data:
   determining, using the one or more processors, a ploidy of the sample, coverage ratio data, allele fraction data, segmentation data, and a copy number model for the one or more gene loci within the one or more subgenomic intervals;
   identifying, using the one or more processors, a plurality of segments based on the segmentation data;
   determining, using the one or more processors, copy numbers for the plurality of segments based on at least the coverage ratio data, the allele fraction data, the segmentation data, and the copy number model;
   detecting, using the one or more processors, the presence of an amplification or a deletion for a gene locus of the one or more gene loci based on the copy number of a corresponding segment of the plurality of segments; and
   calling, using the one or more processors, copy number alterations (CNAs) for the one or more gene loci based on the detected amplifications and deletions for the one or more gene loci.
      2. The method of clause 1, further comprising merging any duplicate amplifications and deletions detected for a gene locus of the one or more gene loci.
      3. The method of clause 1 or clause 2, wherein the copy number model predicts a copy number for the one or more gene loci based on the coverage ratio data and allele fraction data.
      4. The method of any one of clauses 1 to 3, wherein the coverage ratio data further comprises coverage ratio data for single nucleotide polymorphisms (SNPs) and introns associated with the one or more gene loci.
      5. The method of any one of clauses 1 to 4, wherein the copy number model also predicts a sample purity and ploidy for the sample.
      6. The method of any one of clauses 1 to 5, wherein the copy number model also outputs the segmentation data.
      7. The method of any one of clauses 1 to 6, wherein an amplification is detected when the copy number for the corresponding segment is greater than or equal to the ploidy of the sample.
      8. The method of any one of clauses 1 to 7, wherein the detection of deletions comprises identifying homozygous deletions of the one or more gene loci in a corresponding segment.
      9. The method of any one of clauses 1 to 8, wherein the detection of deletions comprises identifying heterozygous deletions of the one or more gene loci in a corresponding segment.
      10. The method of any one of clauses 1 to 9 wherein the detection of deletions comprises identifying partial deletions of the one or more gene loci in a corresponding segment.
      11. The method of any one of clauses 1 to 10, wherein the subject is suspected of having or is determined to have a disease.
      12. The method of clause 11, wherein the disease is cancer.
      13. The method of any one of clauses 1 to 12, wherein the method is used for routine testing.
      14. The method of any one of clauses 1 to 13, wherein the method is used for prenatal testing.
      15. The method of any one of clauses 1 to 14, further comprising collecting the sample from the subject.
      16. The method of any one of clauses 1 to 15, wherein the sample comprises a tissue biopsy sample, a liquid biopsy sample, or a normal control.
      17. The method of clause 16, wherein the sample is a tissue biopsy sample and comprises bone marrow.
      18. The method of clause 16, wherein the sample is a liquid biopsy sample and comprises blood, plasma, cerebrospinal fluid, sputum, stool, urine, or saliva.
      19. The method of clause 16, wherein the sample is a liquid biopsy sample and comprises circulating tumor cells (CTCs).
      20. The method of clause 16, wherein the sample is a liquid biopsy sample and comprises cell-free DNA (cfDNA), circulating tumor DNA (ctDNA), or any combination thereof.
      21. The method of any one of clauses 1 to 20, wherein the plurality of nucleic acid molecules comprises a mixture of tumor nucleic acid molecules and non-tumor nucleic acid molecules.
      22. The method of clause 21, wherein the tumor nucleic acid molecules are derived from a tumor portion of a heterogeneous tissue biopsy sample, and the non-tumor nucleic acid molecules are derived from a normal portion of the heterogeneous tissue biopsy sample.
      23. The method of clause 21, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
      24. The method of any one of clauses 1 to 23, wherein the one or more adapters comprise amplification primers, flow cell adaptor sequences, substrate adapter sequences, or sample index sequences.
      25. The method of any one of clauses 1 to 24, wherein the captured nucleic acid molecules are captured from the amplified nucleic acid molecules by hybridization to one or more bait molecules.
      26. The method of clause 25, wherein the one or more bait molecules comprise one or more nucleic acid molecules, each comprising a region that is complementary to a region of a captured nucleic acid molecule.
      27. The method of any one of clauses 1 to 26, wherein amplifying nucleic acid molecules comprises performing a polymerase chain reaction (PCR) amplification technique, a non-PCR amplification technique, or an isothermal amplification technique.
      28. The method of any one of clauses 1 to 27, wherein the sequencing comprises use of a massively parallel sequencing (MPS) technique, whole genome sequencing (WGS), whole exome sequencing, targeted sequencing, direct sequencing, or Sanger sequencing technique.
      29. The method of clause 28, wherein the sequencing comprises massively parallel sequencing, and the massively parallel sequencing technique comprises next generation sequencing (NGS).
      30. The method of clause 29, wherein the next generation sequencing (NGS) comprises paired end sequencing.
      31. The method of any one of clauses 1 to 30, wherein the sequencer comprises a next generation sequencer.
      32. The method of any one of clauses 1 to 31, further comprising generating, by the one or more processors, a report indicating the called copy number alterations.
      33. The method of clause 32, further comprising transmitting the report to a healthcare provider.
      34. The method of clause 33, wherein the report is transmitted via a computer network or a peer-to-peer connection.
      35. A method for automated calling of copy number alterations comprising:
receiving, at one or more processors, sequence read data for a plurality of sequence reads that overlap one or more gene loci within one or more subgenomic intervals in a sample from a subject, and based on the sequence read data:
   determining, using the one or more processors, a ploidy of the sample, coverage ratio data, allele fraction data, segmentation data, and a copy number model for the one or more gene loci within the one or more subgenomic intervals;
   identifying, using the one or more processors, a plurality of segments based on the segmentation data;
   determining, using the one or more processors, copy numbers for the plurality of segments based on at least the coverage ratio data, the allele fraction data, the segmentation data, and the copy number model;
   detecting, using the one or more processors, the presence of an amplification or a deletion for a gene locus of the one or more gene loci based on the copy number of a corresponding segment of the plurality of segments; and
   calling, using the one or more processors, copy number alterations (CNAs) for the one or more gene loci based on the detected amplifications and deletions for the one or more gene loci.
      36. The method of clause 35, further comprising merging any duplicate amplifications and deletions detected for a gene locus of the one or more gene loci.
      37. The method of clause 35 or clause 36, further comprising generating a report comprising the called copy number alterations for the one or more gene loci.
      38. The method of any one of clauses 35 to 37, further comprising generating a genomic profile for the subject based on the called copy number alterations for the one or more gene loci.
      39. The method of any one of clauses 35 to 38, wherein the coverage ratio data is determined by aligning a plurality of sequence reads that overlap one or more gene loci within one or more subgenomic intervals in the sample and in a control sample to a reference genome, and determining a number of sequence reads that overlap each of the one or more gene loci within the one or more subgenomic intervals in the sample and in the control sample.
      40. The method of clause 39, wherein the control sample is a paired normal sample, a process-matched control sample, or a panel of normal control sample.
      41. The method of any one of clauses 35 to 40, wherein the allele fraction data is determined by aligning a plurality of sequence reads that overlap one or more gene loci within one or more subgenomic intervals in the sample to a reference genome, detecting a number of alleles present at a gene locus of the one or more gene loci, and determining an allele fraction for at least one of the alleles present at the gene locus.
      42. The method of any one of clauses 35 to 41, wherein the segmentation data is generated by: aligning a plurality of sequence reads that overlap one or more gene loci within one or more subgenomic intervals in the sample to a reference genome, and processing the aligned sequence read data, coverage ratio data, and allele fraction data using a pruned exact linear time (PELT) method to determine a number of segments required to account for the aligned sequence read data, wherein each segment has a same copy number.
      43. The method of any one of clauses 35 to 42, wherein the copy number model predicts a copy number for the one or more gene loci based on the coverage ratio data and allele fraction data.
      44. The method of 43, wherein the coverage ratio data further comprises coverage ratio data for single nucleotide polymorphisms (SNPs) and introns associated with the one or more gene loci.
      45. The method of clause 43 or clause 44, wherein the copy number model also predicts a sample purity and ploidy for the sample.
      46. The method of any one of clauses 43 to 45, wherein the copy number model also outputs the segmentation data.
      47. The method of any one of clauses 35 to 46, wherein the ploidy for the sample has a value ranging from 1 to 8.
      48. The method of any one of clauses 35 to 47, wherein an amplification is detected when the copy number for the corresponding segment is greater than or equal to the ploidy of the sample.
      49. The method of clause 48, wherein an amplification is detected when the copy number for the corresponding segment is greater than or equal to the ploidy of the sample plus a first predetermined value.
      50. The method of clause 49, wherein the first predetermined value is a value ranging from 2 to 500.
      51. The method of clause 49 or clause 50, wherein the first predetermined value is a value ranging from 2 to 10.
      52. The method of clause 48, wherein an amplification is detected when the copy number for the corresponding segment is greater than or equal to the ploidy of the sample plus a second predetermined value and the gene locus is a member of a first predefined set of gene loci.
      53. The method of clause 52, wherein the second predetermined value is a value ranging from 0 to 500.
      54. The method of clause 52 or clause 53, wherein the second predetermined value is a value ranging from 2 to 10.
      55. The method of any one of clauses 52 to 54, wherein the first predefined set of gene loci comprises one or more druggable gene target loci, prognostic gene loci, oncogene loci, or any combination thereof.
      56. The method of clause 55, wherein the first predefined set of gene loci comprises the AR and ERBB2 gene loci.
      57. The method of any one of clauses 35 to 56, wherein the detection of deletions comprises identifying homozygous deletions of the one or more gene loci in a corresponding segment.
      58. The method of clause 57, wherein homozygous deletions are detected by determining a total copy number for a given gene locus that is equal to the sum of the copy numbers for a first allele and a second allele at the gene locus.
      59. The method of clause 58, wherein the first allele is a major allele and the second allele is a minor allele.
      60. The method of clause 58 or clause 59, wherein a homozygous deletion is called if the total copy number for a given gene locus is equal to a third predetermined value.
      61. The method of clause 60, wherein the third predetermined value is about zero.
      62. The method of any one of clauses 35 to 61, wherein the detection of deletions comprises identifying heterozygous deletions of the one or more gene loci in a corresponding segment.
      63. The method of clause 62, wherein a heterozygous deletion is called if a copy number for a first allele at a given gene locus is equal to a fourth predetermined value, and a copy number for a second allele at the given gene locus in not equal to the fourth predetermined value.
      64. The method of clause 63, wherein the fourth predetermined value is about zero.
      65. The method of clause 63 or clause 64, wherein the first allele is a major allele and the second allele is a minor allele.
      66. The method of any one of clauses 35 to 65, wherein the detection of deletions comprises identifying partial deletions of the one or more gene loci in a corresponding segment.
      67. The method of clause 66, wherein a partial deletion is called for a given gene locus if log 2 ratios (L2Rs) for neighboring gene loci, single nucleotide polymorphisms (SNPs), and introns are significantly different than the log 2 ratio for the gene locus, and the log 2 ratio for the given gene locus is significantly different from a distribution of L2Rs for non-neighboring gene loci, single nucleotide polymorphisms (SNPs), and introns.
      68. The method of any one of clauses 35 to 67, further comprising performing a quality control procedure prior to calling the copy number alterations for the one or more gene loci.
      69. The method of clause 68, wherein the quality control procedure is performed to assess a quality of the sequence read data.
      70. The method of clause 68 or clause 69, wherein the quality control procedure is performed to assess successful convergence of a copy number model.
      71. The method of any one of clauses 68 to 70, wherein the quality control procedure is performed to assess a reliability of CNA calls for the one or more gene loci.
      72. The method of any one of clauses 35 to 71, wherein the called CNAs are used to diagnose or confirm a diagnosis of disease in the subject.
      73. The method of clause 72, wherein the disease is cancer.
      74. The method of clause 72 or clause 73, further comprising selecting a cancer therapy to administer to the subject based on the called CNAs.
      75. The method of any one of clauses 73 to 74, further comprising determining an effective amount of a cancer therapy to administer to the subject based on the called CNAs.
      76. The method of clause 74 or clause 75, further comprising administering the cancer therapy to the subject based on the called CNAs.
      77. The method of any one of clauses 74 to 76, wherein the cancer therapy comprises chemotherapy, radiation therapy, immunotherapy, a targeted therapy, or surgery.
      78. The method of any one of clauses 74 to 77, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
      79. The method of any one of clauses 35 to 78, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
      80. A method for diagnosing a disease, the method comprising:
diagnosing that a subject has the disease based on detection of copy number alterations (CNAs) for one or more gene loci within one or more subgenomic intervals in a sample from the subject, wherein the detected CNAs are determined according to the method of any one of clauses 35 to 75.
   81. A method of selecting a cancer therapy, the method comprising:
responsive to detecting copy number alterations (CNAs) for one or more gene loci within one or more subgenomic intervals in a sample from a subject, selecting a cancer therapy for the subject, wherein the detected CNAs are determined according to the method of any one of clauses 35 to 79.
   82. A method of treating a cancer in a subject, comprising:
responsive to detecting copy number alterations (CNAs) for one or more gene loci within one or more subgenomic intervals in a sample from a subject, administering an effective amount of a cancer therapy to the subject, wherein the detected CNAs are determined according to the method of any one of clauses 35 to 81.
   83. A method for monitoring tumor progression or recurrence in a subject, the method comprising:
detecting copy number alterations (CNAs) for one or more gene loci within one or more subgenomic intervals in a first sample obtained from the subject at a first time point according to the method of any one of clauses 35 to 81;
detecting copy number alterations (CNAs) for one or more gene loci within one or more subgenomic intervals in a second sample obtained from the subject at a second time point; and
comparing the CNAs detected in the first sample to the CNAs detected in the second sample, thereby monitoring the tumor progression or recurrence.
   84. The method of clause 83, wherein the detection of CNAs in the second sample is determined according to the method of any one of clauses 35 to 81.
   85. The method of clause 83 or clause 84, further comprising adjusting an anti-cancer therapy in response to the tumor progression.
   86. The method of any one of clauses 83 to 85, further comprising adjusting a dosage of the anti-cancer therapy or selecting a different anti-cancer therapy in response to the tumor progression.
   87. The method of clause 86, further comprising administering the adjusted anti-cancer therapy to the subject.
   88. The method of any one of clauses 83 to 87, wherein the first time point is before the subject has been administered an anti-cancer therapy, and wherein the second time point is after the subject has been administered the anti-cancer therapy.
   89. The method of any one of clauses 83 to 88, wherein the subject has a cancer, is at risk of having a cancer, is being routine tested for cancer, or is suspected of having a cancer.
   90. The method of any one of clauses 83 to 89, wherein the cancer is a solid tumor.
   91. The method of any one of clauses 83 to 89, wherein the cancer is a hematological cancer.
   92. The method of any one of clauses 85 to 91, wherein the anti-cancer therapy comprises chemotherapy, radiation therapy, immunotherapy, a targeted therapy, or surgery.
   93. The method of any one of clauses 35 to 79, further comprising determining called CNAs for the one or more gene loci within the one or more subgenomic intervals, and applying the called CNAs as a diagnostic value associated with the sample.
   94. The method of any one of clauses 35 to 79, further comprising generating a genomic profile for the subject based on the called CNAs for the one or more gene loci.
   95. The method of clause 94, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
   96. The method of clause 94 or clause 95, wherein the genomic profile for the subject further comprises results from a nucleic acid sequencing-based test.
   97. The method of any one of clauses 94 to 96, further comprising selecting an anti-cancer agent, administering an anti-cancer agent, or applying an anti-cancer treatment to the subject based on the generated genomic profile.
   98. The method of any one of clauses 35 to 79, wherein the detection of CNAs for the one or more gene loci within one or more subgenomic intervals in the sample is used in making suggested treatment decisions for the subject.
   99. The method of any one of clauses 35 to 79, wherein the detection of CNAs for the one or more gene loci within one or more subgenomic intervals in the sample is used in applying or administering a treatment to the subject.
   100. A system comprising:
one or more processors; and
a memory communicatively coupled to the one or more processors and configured to store instructions that, when executed by the one or more processors, cause the system to:
   receive sequence read data for a plurality of sequence reads that overlap one or more gene loci within one or more subgenomic intervals in a sample from a subject, and based on the sequence read data:
   determine a ploidy of the sample, coverage ratio data, allele fraction data, segmentation data, and copy number model for the one or more gene loci within the one or more subgenomic intervals;
   identify a plurality of segments based on the segmentation data;
   determine copy numbers for the plurality of segments based on at least the coverage ratio data, the allele fraction data, the segmentation data, and the copy number model;
   detect the presence of an amplification or a deletion for a gene locus of the one or more gene loci based on the copy number of a corresponding segment of the plurality of segments; and
   call copy number alterations (CNAs) for the one or more gene loci based on the detected amplifications and deletions for the one or more gene loci.
      101. A non-transitory computer-readable storage medium storing one or more programs, the one or more programs comprising instructions, which when executed by one or more processors of a system, cause the system to:
receive sequence read data for a plurality of sequence reads that overlap one or more gene loci within one or more subgenomic intervals in a sample from a subject, and based on the sequence read data:
determine a ploidy of the sample, coverage ratio data, allele fraction data, segmentation data, and a copy number model for the one or more gene loci within the one or more subgenomic intervals in a sample from a subject;
identify a plurality of segments based on the segmentation data;
determine copy numbers for the plurality of segments based on at least the coverage ratio data, the allele fraction data, the segmentation data, and the copy number model;
detect the presence of an amplification or deletion for a gene locus of the one or more gene loci based on a copy number of a corresponding segment of the plurality of segments; and
call copy number alterations (CNAs) for the one or more gene loci based on the detected amplifications and deletions for the one or more gene loci.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method for automated calling of copy number alterations comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules, wherein one or more of the plurality of sequencing reads overlap one or more gene loci within one or more subgenomic intervals in the sample;
receiving, at one or more processors, sequence read data for the plurality of sequence reads that overlap one or more gene loci within one or more subgenomic intervals in a sample from the subject, and based on the sequence read data:
determining, using the one or more processors, a ploidy of the sample, coverage ratio data, allele fraction data, segmentation data, and a copy number model for the one or more gene loci within the one or more subgenomic intervals;
identifying, using the one or more processors, a plurality of segments based on the segmentation data;
determining, using the one or more processors, copy numbers for the plurality of segments based on at least the coverage ratio data, the allele fraction data, the segmentation data, and the copy number model;
detecting, using the one or more processors, the presence of an amplification or a deletion for a gene locus of the one or more gene loci based on the copy number of a corresponding segment of the plurality of segments; and
calling, using the one or more processors, copy number alterations (CNAs) for the one or more gene loci based on the detected amplifications and deletions for the one or more gene loci.

2. The method of claim 1, further comprising merging any duplicate amplifications and deletions detected for a gene locus of the one or more gene loci.

3.-4. (canceled)

5. The method of claim 1, wherein the coverage ratio data is determined by aligning a plurality of sequence reads that overlap one or more gene loci within one or more subgenomic intervals in the sample and in a control sample to a reference genome, and determining a number of sequence reads that overlap each of the one or more gene loci within the one or more subgenomic intervals in the sample and in the control sample.

6. The method of claim 5, wherein the control sample is a paired normal sample, a process-matched control sample, or a panel of normal control sample.

7. The method of claim 1, wherein the allele fraction data is determined by aligning a plurality of sequence reads that overlap one or more gene loci within one or more subgenomic intervals in the sample to a reference genome, detecting a number of alleles present at a gene locus of the one or more gene loci, and determining an allele fraction for at least one of the alleles present at the gene locus.

8. The method of claim 1, wherein the segmentation data is generated by:
aligning a plurality of sequence reads that overlap one or more gene loci within one or more subgenomic intervals in the sample to a reference genome, and
processing the aligned sequence read data, coverage ratio data, and allele fraction data using a pruned exact linear time (PELT) method to determine a number of segments required to account for the aligned sequence read data, wherein each segment has a same copy number.

9. The method of claim 1, wherein the copy number model predicts a copy number for the one or more gene loci based on the coverage ratio data and allele fraction data.

10. The method of claim 9, wherein the coverage ratio data further comprises coverage ratio data for single nucleotide polymorphisms (SNPs) and introns associated with the one or more gene loci.

11. The method of claim 9, wherein the copy number model also predicts a sample purity and ploidy for the sample.

12. The method of claim 9, wherein the copy number model also outputs the segmentation data.

13. The method of claim 1, wherein the ploidy for the sample has a value ranging from 1 to 8.

14. The method of claim 1, wherein an amplification is detected when the copy number for the corresponding segment is greater than or equal to the ploidy of the sample.

15. The method of claim 14, wherein an amplification is detected when the copy number for the corresponding segment is greater than or equal to the ploidy of the sample plus a first predetermined value.

16.-17. (canceled)

18. The method of claim 14, wherein an amplification is detected when the copy number for the corresponding segment is greater than or equal to the ploidy of the sample plus a second predetermined value and the gene locus is a member of a first predefined set of gene loci.

19.-20. (canceled)

21. The method of claim 18, wherein the first predefined set of gene loci comprises one or more druggable gene target loci, prognostic gene loci, oncogene loci, or any combination thereof.

22. (canceled)

23. The method of claim 1, wherein the detection of deletions comprises identifying homozygous deletions of the one or more gene loci in a corresponding segment.

24. The method of claim 23, wherein homozygous deletions are detected by determining a total copy number for a given gene locus that is equal to the sum of the copy numbers for a first allele and a second allele at the gene locus.

25. The method of claim 24, wherein the first allele is a major allele and the second allele is a minor allele.

26. The method of claim 24, wherein a homozygous deletion is called if the total copy number for a given gene locus is equal to a third predetermined value.

27. The method of claim 26, wherein the third predetermined value is about zero.

28. The method of claim 1, wherein the detection of deletions comprises identifying heterozygous deletions of the one or more gene loci in a corresponding segment.

29. The method of claim 28, wherein a heterozygous deletion is called if a copy number for a first allele at a given gene locus is equal to a fourth predetermined value, and a copy number for a second allele at the given gene locus in not equal to the fourth predetermined value.

30. The method of claim 29, wherein the fourth predetermined value is about zero.

31. The method of claim 29, wherein the first allele is a major allele and the second allele is a minor allele.

32. The method of claim 1, wherein the detection of deletions comprises identifying partial deletions of the one or more gene loci in a corresponding segment.

33. The method of claim 32, wherein a partial deletion is called for a given gene locus if log 2 ratios (L2Rs) for neighboring gene loci, single nucleotide polymorphisms (SNPs), and introns are significantly different than the log 2 ratio for the gene locus, and the log 2 ratio for the given gene locus is significantly different from a distribution of L2Rs for non-neighboring gene loci, single nucleotide polymorphisms (SNPs), and introns.

34. (canceled)

35. The method of claim 1, further comprising performing a quality control procedure prior to calling the copy number alterations for the one or more gene loci, wherein the quality control procedure is performed to assess a quality of the sequence read data, assess successful convergence of a copy number model, or assess a reliability of CNA calls for the one or more gene loci.

36.-37. (canceled)

38. The method of claim 1, wherein the called CNAs are used to diagnose or confirm a diagnosis of cancer in the subject.

39. (canceled)
